(19) United States
(12) Patent Application Publication
(10) Pub. No.: US 2016/0081665 A1
(43) Pub. Date: Mar. 24, 2016

(54) ULTRASONIC PROBE ULTRASONIC IMAGING APPARATUS AND CONTROLLING METHOD OF ULTRASONIC IMAGING APPARATUS

(71) Applicant: SAMSUNG ELECTRONICS CO., LTD., Suwon-si (KR)

(72) Inventors: SUNGCHAN PARK, Suwon-si (KR); JUNGHO KIM, Yongin-si (KR); Kyuhong KIM, Seoul (KR); Bae hyung KIM, Yongin-si (KR); Jooyoung KANG, Yongin-si (KR)

(73) Assignee: SAMSUNG ELECTRONICS CO., LTD., Suwon-si (KR)

(21) Appl. No.: 14/805,943

(22) Filed: Jul. 22, 2015

(30) Foreign Application Priority Data
Sep. 23, 2014 (KR) 10-2014-0127008

(51) Int. Cl. A61B 8/08; A61B 8/14; A61B 8/00

(52) U.S. Cl. CPC A61B 8/5269; A61B 8/4483; A61B 8/14; A61B 8/54; A61B 8/5207

(57) ABSTRACT

An ultrasonic probe includes a transducer circuit configured to receive ultrasonic waves reflected from an object and output an ultrasonic signal based on the ultrasonic waves; a simulated circuit having circuit characteristics substantially the same as the transducer circuit and configured to output a noise signal; and a differential amplifier circuit configured to output a voltage difference between the ultrasonic signal and the noise signal.

### CROSS-REFERENCE TO RELATED APPLICATION

This application claims priority from Korean Patent Application No. 10-2014-0127008, filed on Sep. 23, 2014, in the Korean Intellectual Property Office, the disclosure of which is incorporated herein by reference in its entirety.

### BACKGROUND

1. Field

Apparatuses and methods consistent with exemplary embodiments relate to transmitting and receiving ultrasonic waves by using an ultrasonic probe in an ultrasonic imaging apparatus.

2. Description of Related Art

An ultrasonic imaging apparatus transmits ultrasonic waves to a target part in an object through the surface of the object, detects echo ultrasonic waves reflected from the object and noninvasively provides images about an examined part, such as a tomogram of a soft tissue or bloodstream.

An ultrasonic imaging apparatus is compact, inexpensive, and quickly displays a diagnostic image as compared with other types of diagnostic imaging apparatuses, e.g., an X-ray apparatus, a computed tomography (CT) scanner, a magnetic resonance imaging (MRI) apparatus, and a diagnostic nuclear medical apparatus. In addition, the ultrasonic imaging apparatus is safe because there is no risk of radiation exposure. Therefore, the ultrasonic imaging apparatus is widely used in medical examination for maternity, cardiology, abdomen, and urology clinics.

An ultrasonic probe included in an ultrasonic imaging apparatus transmits ultrasonic waves to an object, receives ultrasonic waves reflected from the object, and converts the received ultrasonic waves into electrical signals.

### SUMMARY

One or more exemplary embodiments provide an ultrasonic probe capable of outputting ultrasonic signals, in which noise signals are removed, when receiving ultrasonic waves reflected from an object.

One or more exemplary embodiments also provide an ultrasonic imaging apparatus capable of removing noise signals caused by power and generating ultrasonic images based on ultrasonic signals from which the noise signals are removed, and a method of controlling the same.

Additional aspects of the present disclosure will be set forth in part in the description which follows and, in part, will be obvious from the description, or may be learned by practice of the exemplary embodiments.

According to an aspect of an exemplary embodiment, an ultrasonic probe includes: a transducer circuit configured to receive ultrasonic waves reflected from an object and output an ultrasonic signal based on the ultrasonic waves; a simulated circuit having circuit characteristics substantially the same as the transducer circuit and configured to output a noise signal; and a differential amplifier circuit configured to output a voltage difference between the ultrasonic signal and the noise signal.

The transducer circuit and the simulated circuit may be supplied with a voltage from a same power source.

The simulated circuit may include one or more electrical elements, and may output the noise signal based on a variable control configured to control at least one value of the one or more electrical elements.

The differential amplifier circuit may amplify the voltage difference between the ultrasonic signal of the transducer circuit and the noise signal of the simulated circuit.

The ultrasonic probe may further include a low-noise amplifier circuit configured to reduce at least one of noise included in the ultrasonic signal of the transducer circuit and the noise signal of the simulated circuit.

The transducer circuit may include a transducer module configured to emit the ultrasonic waves to the object and receive the ultrasonic waves reflected from the object.

According to an aspect of another exemplary embodiment, an ultrasonic imaging apparatus includes: a display; an ultrasonic probe configured to output a voltage difference between an ultrasonic signal of a transducer circuit and a noise signal of a simulated circuit, the transducer circuit receiving ultrasonic waves reflected from an object and outputting the ultrasonic signal based on the ultrasonic waves, and the simulated circuit having circuit characteristics substantially the same as the transducer circuit and outputting the noise signal; and a controller configured to control the ultrasonic probe, filter noise from the ultrasonic signal based on the noise signal, generate an ultrasonic image from the filtered ultrasonic signal, and control the display to display the ultrasonic image.

The controller may include a simulated circuit controller configured to control the simulated circuit.

The ultrasonic imaging apparatus may further include a power source configured to supply a voltage to the transducer circuit and the simulated circuit.

The simulated circuit may include one or more electrical elements, and may output the noise signal based on a variable control configured to control at least one value of the one or more electrical elements.

The ultrasonic imaging apparatus may further include a differential amplifier circuit configured to amplify the voltage difference.

The ultrasonic imaging apparatus may further include a low-noise amplifier circuit configured to reduce at least one of noise included in the first ultrasonic signal of the transducer circuit and the noise signal of the simulated circuit.

The controller may include an ultrasonic generating controller configured to control the transducer circuit to emit the ultrasonic waves; and an ultrasonic receiving controller configured to generate a reconstructed ultrasonic signal based on the voltage difference.

The transducer circuit may include a transducer module configured to emit the ultrasonic waves to the object and receive the ultrasonic waves from the object.

The controller may further include an imaging processor configured to generate the ultrasonic image based on the reconstructed ultrasonic signal.

According to an aspect of still another exemplary embodiment, a method of controlling an ultrasonic imaging apparatus includes: supplying a voltage to a transducer circuit and a simulated circuit, the simulated circuit having circuit characteristics substantially the same as the transducer circuit; receiving ultrasonic waves reflected from an object; outputting, by the transducer circuit, an ultrasonic signal based on the ultrasonic waves, and outputting, by the simulated circuit, a noise signal; outputting a voltage difference between the ultrasonic signal of the transducer circuit and the noise signal of the simulated circuit; and filtering noise from the ultrasonic signal based on the noise signal and generating an ultrasonic image from the filtered ultrasonic signal.

The control method may further include controlling at least one value of one or more electrical elements of the simulated circuit.

The method may further include emitting the ultrasonic waves to the object through the transducer; and receiving the ultrasonic waves reflected from the object.

The outputting the voltage difference may include amplifying the voltage difference.

### BRIEF DESCRIPTION OF THE DRAWINGS

The above and/or other aspects will become more apparent by describing certain exemplary embodiments with reference to the accompanying drawings, in which:

FIG. 1 is a perspective view illustrating an exemplary configuration of an ultrasonic imaging apparatus;

FIG. 2 is a block diagram illustrating an exemplary configuration of an ultrasonic imaging apparatus;

FIG. 3 is a block diagram illustrating a probe module according to an exemplary embodiment;

FIG. 4 is a view illustrating a process of transmitting and receiving ultrasonic waves of a probe module according to an exemplary embodiment;

FIG. 5 is a circuit diagram of a transducer circuit according to an exemplary embodiment;

FIG. 6 is a view conceptually illustrating a configuration of a transducer array of a transducer module according to an exemplary embodiment;

FIG. 7 is an equivalent circuit diagram of a transducer module of an ultrasonic probe according to an exemplary embodiment;

FIG. 8 is a view illustrating a simulated circuit according to an exemplary embodiment;

FIG. 9 is an equivalent circuit diagram of a variable control module according to an exemplary embodiment;

FIG. 10A is an example of an equivalent circuit diagram of a transducer circuit;

FIGS. 10B and 10C are examples of an equivalent circuit diagram of a simulated circuit;

FIGS. 11A and 11B are circuit diagrams of a probe module including a plurality of transducer circuits according to exemplary embodiments; and

FIG. 12 is a control flowchart of an ultrasonic imaging apparatus according to an exemplary embodiment.

### DETAILED DESCRIPTION

Exemplary embodiments are described in greater detail with reference to the accompanying drawings.

In the following description, the same drawing reference numerals are used for the same elements even in different drawings. The matters defined in the description, such as detailed construction and elements, are provided to assist in a comprehensive understanding of the exemplary embodiments. Thus, it is apparent that the exemplary embodiments can be carried out without those specifically defined matters. Also, well-known functions or constructions are not described in detail since they may obscure the exemplary embodiments with unnecessary detail. It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section.

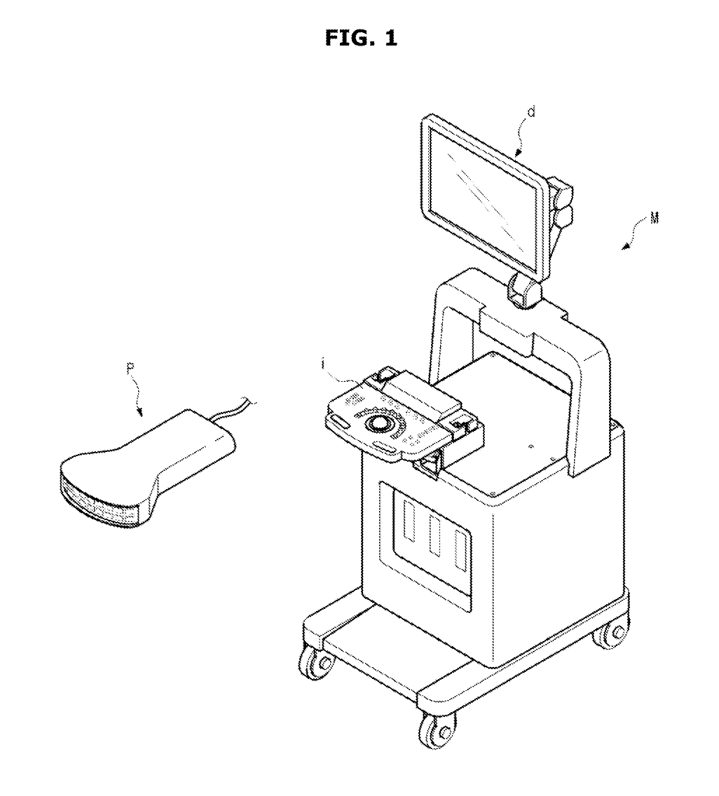

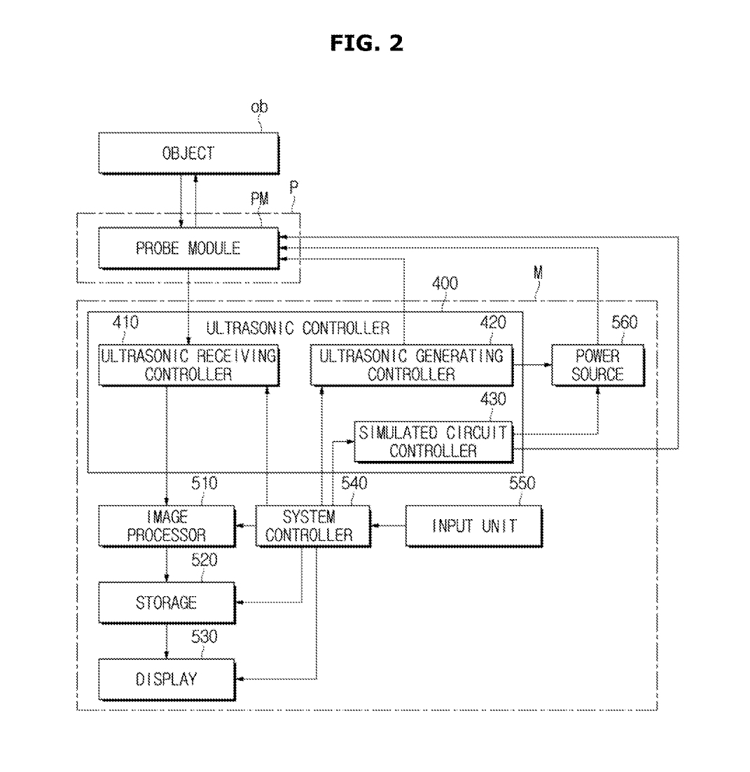

FIG. 1 is a perspective view illustrating an exemplary configuration of an ultrasonic imaging apparatus, and FIG. 2 is a block diagram illustrating an exemplary configuration of an ultrasonic imaging apparatus.

As illustrated in FIGS. 1 and 2, an ultrasonic imaging apparatus may include an ultrasonic probe (P) emitting ultrasonic waves to an object (ob), receiving ultrasonic waves reflected from the object (ob), and converting received ultrasonic waves into electrical signals, and a main body (M) generating ultrasonic images based on ultrasonic signals. As illustrated in FIG. 1, the main body (M) may be connected to the ultrasonic probe (P), and may be a workstation provided with an input unit (i) and a display (d).

As illustrated in FIG. 2, the ultrasonic probe (P) may include a probe module (PM) collecting information about a target part of an object (ob) by using ultrasonic waves, and the probe module (PM) will be described later in detail.

The main body (M) may include an image processor 510 generating images based on ultrasonic signals output from the ultrasonic probe (P), a storage 520 storing ultrasonic images generated in the image processor 510, and a display 530 displaying the ultrasonic images.

The main body (M) may include an ultrasonic controller 400 controlling transmission and reception of ultrasonic waves of the probe module (PM), a power source 560 applying a certain amount of an alternating current (AC) to the probe module (PM), an input unit 550 receiving a certain command from a user to control the ultrasonic imaging apparatus, and a system controller 540 controlling operations of the ultrasonic imaging apparatus. The ultrasonic controller 400 may include an ultrasonic receiving controller 410, an ultrasonic generating controller 420, and a simulated circuit controller 430. Hereinafter, components of the main body (M) will be described.

The image processor 510 may generate images of an object (ob) based on reconstructed ultrasonic signals received from the ultrasonic receiving controller 410 so that a user, such as a doctor and/or a patient, may visually examine the object (ob), such as the inside of a human body. The reconstructed ultrasonic signals may be output from the ultrasonic receiving controller 410 to the image processor 510 to generate ultrasonic images. In addition, the image processor 510 may transmit the ultrasonic image generated based on the reconstructed ultrasonic image of the image processor 510 to the storage 520 and the display 530.

The image processor 510 may perform additional image processing for ultrasonic images. For example, the image processor 510 may perform post-processing, such as correcting or adjusting contrast, brightness, and/or sharpness of ultrasonic images.

In addition, the image processor 510 may emphasize a target part by coloring the target part in a particular color or indicating the target part with a marker to distinguish the target part from other parts in an ultrasonic image. The image processor 510 may generate a three dimensional ultrasonic image by using a plurality of ultrasonic images after generating the plurality of ultrasonic images. The additional processing of images by the image processor 510 may be performed according to a predetermined setting or according to a command from a user input through the input unit 550.

The storage 520 may store ultrasonic images generated in the image processor 510 and ultrasonic images upon which the post-processing is performed. The display 530 may display the ultrasonic images generated by the image processor 510 and ultrasonic images stored in the storage 520 so that a user may visually examine a structure and organization of the inside of the object (ob).

The storage 520 may store programs related to functions of the ultrasonic imaging apparatus and information of patients input to the ultrasonic imaging apparatus, and the generated ultrasonic images. The storage 520 may include a program part and a data part.

The program part may store operating system (OS) booting programs to control general operations of the ultrasonic imaging apparatus. The program part may include programs related to operations of the system controller 540.

The data part may store data generated according to the use of the ultrasonic imaging apparatus. Particularly, the data part may store ultrasonic images generated by the image processor 510 and information of patients input through the input unit 550 by a user.

The storage 520 may include, but is not limited to, a nonvolatile memory, such as, a cache memory, a read only memory (ROM), a programmable ROM (PROM), an erasable programmable ROM (EPROM), an electrically erasable programmable ROM (EEPROM), and a flash memory, volatile memory, such as, a random access memory (RAM), a hard disk drive (HDD), or compact disc (CD)-ROM.

The display 530 may display ultrasonic images in two dimensions or three dimensions, which are generated by the image processor 510 and stored in the storage 520.

The display 530 may include, for example, a plazma display panel (PDP), a light emitting diode (LED), or a liquid crystal display (LCD). In addition, the display 530 may employ a three dimensional display device capable of displaying a three dimensional image. The display 530 may include a touch screen. When the display 530 includes the touch screen, the display 530 may perform as the input unit 550. The touchscreen may employ a resistive touchscreen panel or a capacitive touchscreen panel. Otherwise, the touch screen may use ultrasonic waves or infrared light.

The ultrasonic generating controller 420 may generate pulses according to a command from the system controller 540, and may transmit the pulses to a transducer circuit (T) in the probe module (PM). The probe module (PM) may generate ultrasonic waves according to the pulses output from the ultrasonic generating controller 420 and may irradiate the ultrasonic waves to the object (ob).

The ultrasonic generating controller 420 may generate a control signal for the power source 560 so that the power source 560 may apply a certain amount of AC or direct current (DC) to the probe module (PM).

The simulated circuit controller 430 may generate variable control signals according to a command from the system controller 540 described later, or a command input through the input unit 550 by a user. The simulated circuit controller 430 may transmit the variable control signals to a simulated circuit (S) in the probe module (PM). According to the variable control signals output from the simulated circuit controller 430 of the probe module (PM), the probe module (PM) may control (or change) values of elements, such as a resistance, an inductance value, or a capacitance value of elements, such as a resistor, an inductor, or a capacitor, in the probe module (PM).

Likewise, the ultrasonic generating controller 420, the simulated circuit controller 430 may generate an additional control signal for the power source 560 so that the power source 560 may apply a certain amount of AC or DC to the probe module (PM).

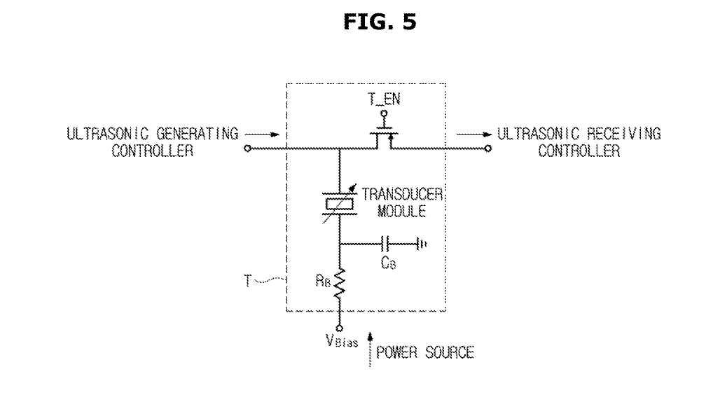

The ultrasonic receiving controller 410 may generate a reconstructed ultrasonic signal based on an ultrasonic signal output from the probe module (PM) according to a command from the system controller 540. The reconstructed ultrasonic signal may be supplied to the image processor 510 to be used when generating an ultrasonic image of the image processor 510. In addition, the ultrasonic receiving controller 410 may further output a control signal for determining whether to receive an ultrasonic signal output from the transducer circuit (T) in the probe module (PM). The control signal for determining whether to receive the ultrasonic signal will be described later with reference to FIG. 5.

The system controller 540 may control overall operations of the ultrasonic imaging apparatus, such as the ultrasonic generating controller 420, the simulated circuit controller 430, the ultrasonic receiving controller 410, the image processor 510, the storage 520, and the display 530.

For example, the system controller 540 may control operations of the ultrasonic imaging apparatus according to a predetermined setting, or may control operations of the ultrasonic imaging apparatus by generating a certain control command according to an instruction or command through the input unit 550 from a user.

The input unit 550 may receive a certain instruction or command from a user to control the ultrasonic imaging apparatus. The input unit 550 may include, but is not limited to, a user interface, such as a keyboard, a mouse, a trackball, a touch screen, a paddle.

The power source 560 may supply AC or AC voltage to each element of the main body (M), and may apply AC or AC voltage to the probe module (PM) according to the control of the ultrasonic controller 400. For example, the power source 560 may be commercial AC power, an external power source, an internal capacitor, or a battery. Hereinafter, the AC or the AC voltage supplied from the power source 560 may be referred to as V_{Bias}.

As mentioned above, elements of the main body (M) may perform control operations to transmit or receive ultrasonic waves to or from the object, such as controlling the probe module (PM) included in the ultrasonic probe (P), applying a voltage, or receiving a selection from a user.

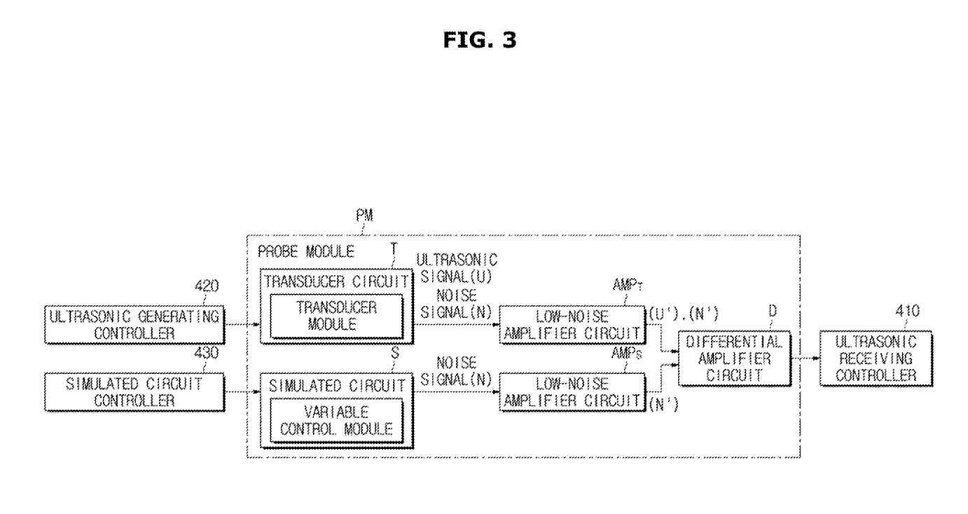

Hereinafter, an ultrasonic probe (P) included in an ultrasonic imaging apparatus will be described. The ultrasonic probe (P) may collect information about a target part of an object (ob) by using ultrasonic waves. Referring to FIG. 2, the ultrasonic probe (P) may include the probe module (PM) to transmit and receive ultrasonic waves, as illustrated in FIG. 1. Hereinafter, a control operation of the probe module (PM) will be described with reference to FIGS. 3 to 5.

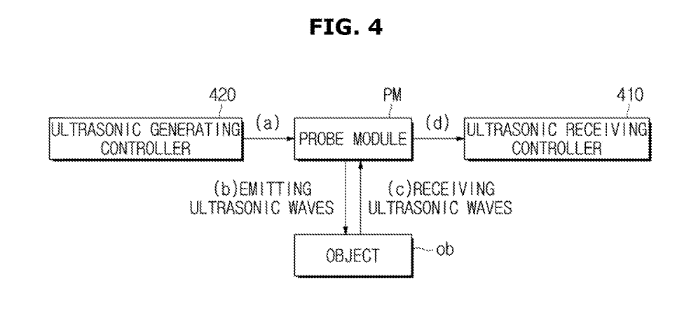

FIG. 3 is a block diagram illustrating a probe module (PM) according to an exemplary embodiment, FIG. 4 is a view illustrating a process of transmitting and receiving ultrasonic waves of a probe module (PM), and FIG. 5 is a circuit diagram of a transducer circuit (T).

Referring to FIG. 3, the probe module (PM) may include a transducer circuit (T) irradiating ultrasonic waves to a target part inside an object (ob) by generating ultrasonic waves, receiving ultrasonic waves reflected from the object (ob), and converting the ultrasonic waves into an electrical signal (or an ultrasonic signal), a simulated circuit (S) including the same (or substantially the same) circuit characteristic as the transducer circuit (T), low-noise amplifier circuits (AMP_{T} and AMP_{S}) removing noise signals output from the transducer circuit (T) and the simulated circuit (S), respectively and a differential amplifier circuit (D) amplifying a voltage difference between output signals output from the low-noise amplifier circuits (AMP_{T} and AMP_{S}s). Hereinafter, a process of transmitting and receiving ultrasonic waves of a probe module (PM) will be described with reference to FIGS. 3 and 4.

Referring to FIGS. 3 and 4, the ultrasonic generating controller 420 may apply pulses to the transducer circuit (T) in the probe module (PM) (operation a). The transducer circuit (T) may generate ultrasonic waves based on the pulse applied from the ultrasonic generating controller 420, and may irradiate the generated ultrasonic waves to an object (ob) (operation b). The ultrasonic waves irradiated to the object (ob) may be reflected from a target part inside the object (ob). The transducer circuit (T) may receive ultrasonic waves reflected from the object (ob) (operation c), may convert the ultrasonic waves into an electrical signal (an ultrasonic signal), and may output the electrical signal (operation d). Hereinafter, the transducer circuit (T) will be described in detail with reference to FIG. 5.

Referring to FIG. 5, the transducer circuit (T) may include a transducer module converting ultrasonic waves into electrical signals, and other elements R_{B} and C_{B}. The transducer circuit (T) may be supplied with a voltage (V_{Bias}) from the power source 560 of the main body (M), and when being supplied with a voltage (V_{Bias}), a piezoelectric vibrator or a thin film included in the transducer module in the transducer circuit (T) may vibrate.

The transducer module may emit ultrasonic waves to an object. The ultrasonic waves may be generated by the piezoelectric vibrator or vibrating thin film according to a control signal of the ultrasonic generating controller 420. When the transducer module may receive the reflected ultrasonic waves reflected from the object, the piezoelectric vibrator or the thin film constituting the transducer module may vibrate corresponding to the received ultrasonic waves. The transducer module may generate DC frequencies corresponding to a vibration frequency of the piezoelectric vibrator or the thin film, and may convert the ultrasonic waves into electrical signals (or ultrasonic signals). The ultrasonic signals may be output to the ultrasonic receiving controller 410 depending on a control signal (T_EN) of the transducer circuit (T).

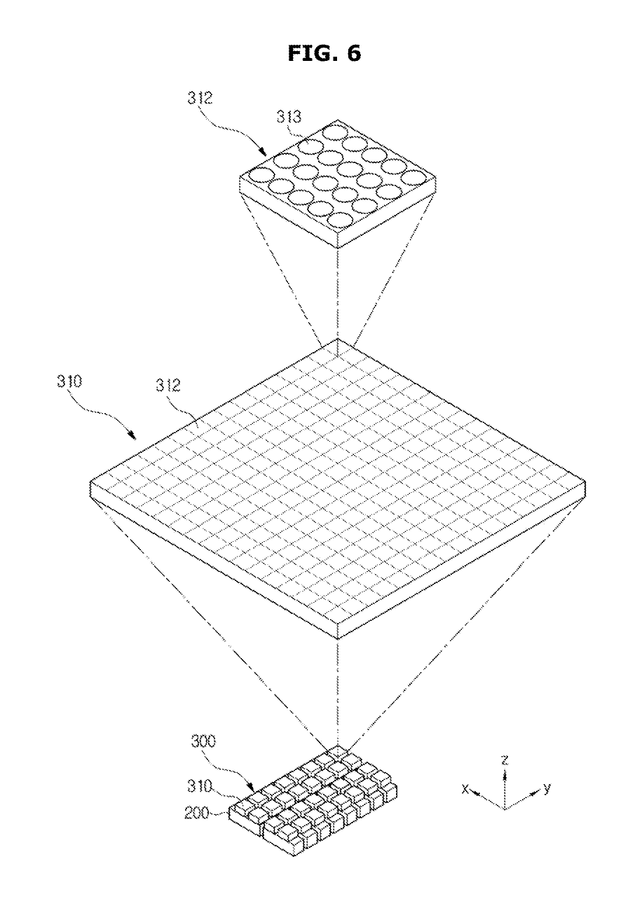

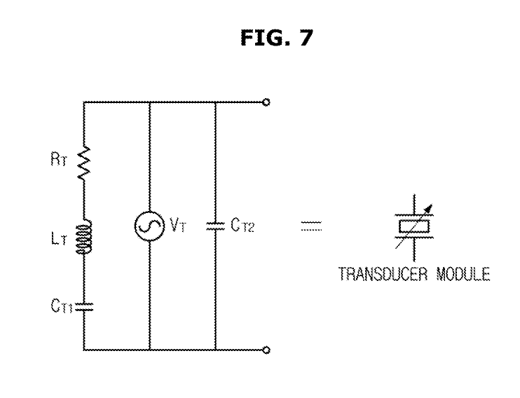

Hereinafter, a transducer module may be described in greater detail with reference to FIGS. 6 and 7. FIG. 6 is a view conceptually illustrating a configuration of a transducer array of a transducer module according to an exemplary embodiment, and FIG. 7 is an equivalent circuit diagram of a transducer module of an ultrasonic probe according to an exemplary embodiment.

Referring to FIG. 6, a transducer may include a transducer array 300 sending and/or receiving ultrasonic waves, and an integrated circuit 200 in which the transducer array 300 is mounted.

The transducer array 300 may include a plurality of transducer elements 312 sending and/or receiving ultrasonic waves. The transducer element 312 may include, but is not limited to, magnetostrictive ultrasonic transducers that convert wave energy into electricity energy using the magnetostrictive effect of a magnetic material, piezoelectric ultrasonic transducers using the piezoelectric effect of a piezoelectric material, piezoelectric micromachined ultrasonic transducers (pMUTs), or capacitive micromachined ultrasonic transducers (cMUTs) that transmit and/or receive ultrasonic waves using vibration of several hundreds or thousands of micromachined thin films. In an exemplary embodiment, cMUTs may be described as an example of the transducer element 312.

As illustrated in FIG. 6, the cMUT array 300 may be formed in a two dimensional array in which a plurality of tiles 310 are arranged in columns and rows. The tile 310 may be a basic unit forming the cMUT array 300. The tile 310 may be formed with the transducer element 312 arranged in the two dimensional array. The transducer element 312 may include a plurality of thin films arranged in the two dimensional array, and the thin film may vibrate when electrical signals are applied.

For example, the cMUT array 300 may have a size of 4 by 8, which contains 32 tiles 310. In addition, a single tile 310 may be formed in an array having a size of 16 by 16, which contains 256 of the transducer element 312. A single transducer element 312 may include approximately 20 to 25 of thin films 313. In this case, the cMUT array 300 may include approximately 163,840 to 204,800 of the thin films 313 in total.

As mentioned above, when the cMUT array 300 of the ultrasonic probe (P) is formed in a two dimensional array having a size of 4 by 8, which contains 32 of tiles 310, the cMUT array 300 may be mounted to integrated circuits 200 to control electrical signals applied to two tiles 310 disposed on an upper portion of each column of the cMUT array 300, and to two tiles 310 disposed on a lower portion of each column of the cMUT array 300, respectively.

When a transmit signal is applied, the integrated circuit 200 may adjust generation of ultrasonic waves by controlling the transmit signal applied to the cMUT array 300 according to logic. The applied signal may be transmit pulses output from the ultrasonic generating controller 420 of the main body (M).

The configuration of the transducer module may be an equivalent circuit diagram, as illustrated in FIG. 7. The transducer module may correspond to an equivalent circuit diagram including a resistor (R_{T}), an inductor (L_{T}), and capacitors (C_{T1} and C_{T2}). When receiving ultrasonic waves, an ultrasonic signal (V_{T}) output from the transducer module may be applied to the low-noise amplifier circuit (AMP_{T}), as illustrated in FIG. 3.

Because the transducer circuit (T) may include a transducer module supplied with a voltage (V_{Bias}) from the power source 560 of the main body (M) and other elements (R_{B} and C_{B}), the transducer circuit (T) may further output noise signals produced by elements of the transducer module (e.g., the resistor (R_{T}), the inductor (L_{T}), and the capacitors (C_{T1} and C_{T2})) and other elements (R_{B} and C_{B}), hereinafter collectively referred to as “power terminal.” The noise signal may be applied to the low-noise amplifier circuit (AMP_{T}).

Therefore, when receiving ultrasonic waves, the transducer circuit (T) may output both an ultrasonic signal from the transducer module and a noise signal from the power terminal of the transducer circuit (T).

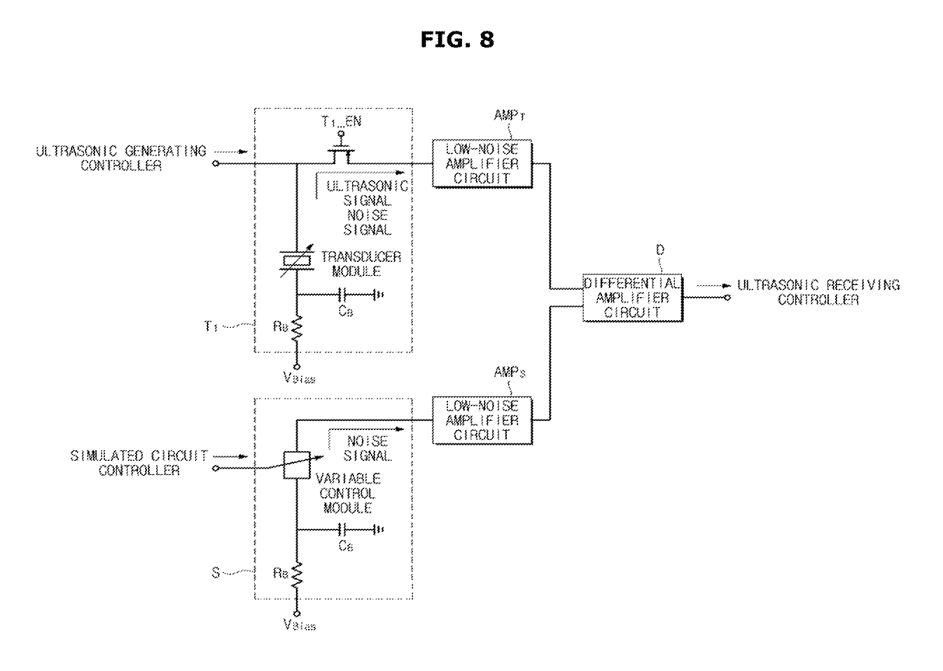

In accordance with an exemplary embodiment, the probe module (PM) may include a simulated circuit (S) to remove or reduce the noise signal output from the transducer circuit (T). The simulated circuit (S) may be a circuit having the circuit characteristics the same as the transducer circuit (T) and will be described later with reference to FIG. 8.

Referring to FIG. 3 again, the simulated circuit controller 430 may apply a pulse (hereinafter referred to as a variable control signal) to the simulated circuit (S) in the probe module (PM). The simulated circuit (S) may control an element value of a variable control module in the simulated circuit (S), which will be described later, based on the variable control signal applied from the simulated circuit controller 430.

FIG. 8 is a view illustrating a simulated circuit (S) according to an exemplary embodiment.

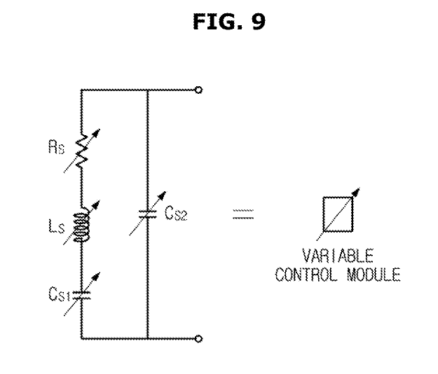

Referring to FIG. 8, the simulated circuit (S) may include a variable control module and other elements (R_{B} and C_{B}). The simulated circuit (S) may be supplied with a voltage (V_{Bias}) from the power source 560 of the main body (M), similar to the transducer circuit (T). The voltage (V_{Bias}) supplied to the simulated circuit (S) may be the same as that of the transducer circuit (T). Hereinafter, the variable control module and the simulated circuit (S) will be described in greater detail with reference to FIGS. 8 and 9. FIG. 9 is an equivalent circuit diagram of a variable control module according to an exemplary embodiment.

Referring to FIGS. 8 and 9, the simulated circuit (S) may be supplied with a voltage (V_{Bias}) from the power source 560 of the main body (M), and may be realized as a circuit diagram having the circuit characteristics the same as the transducer circuit (T). The simulated circuit (S) may output noise signals generated by a variable control module including a resistor (R_{S}), an inductor (L_{S}), and capacitors (C_{S1} and C_{S2})) and other elements (R_{B} and C_{B}), hereinafter collectively referred to as “power terminal,” to a low-noise amplifier circuit (AMP_{S}). The simulated circuit (S) and the transducer circuit (T) may have the same circuit characteristics, and may be supplied with a voltage (V_{Bias}) from the same power source 560. Therefore, the noise signal output from the simulated circuit (S) may be the same as that of the transducer circuit (T).

For example, when the transducer circuit (T) includes the resistor (R_{B}) and the capacitor (C_{B}), in which the resistor (R_{B}) is connected between the transducer module and the voltage (V_{Bias}), and the capacitor (C_{B}) is connected to a node between the transducer module and the resistor (R_{B}), and ground, the simulated circuit (S) may include the resistor (R_{B}) and the capacitor (C_{B}) in the power terminal, in which the resistor (R_{B}) may be connected between the variable control module and the voltage (V_{Bias}) and the capacitor (C_{B}) may be connected to a node between the variable control module and the resistor (R_{B}), and ground, as illustrated in FIG. 8.

Because the simulated circuit (S) may not include a transducer module, unlike the transducer circuit (T), the simulated circuit (S) may not receive ultrasonic waves, and may output only noise signals. That is, the simulated circuit (S) may be realized by an equivalent circuit diagram of the transducer circuit (T) applied with a current or a voltage (V_{Bias}) from the power 560 in a state in which ultrasonic waves are not received.

Referring to FIG. 9, the simulated circuit controller 430 may control element values (e.g., resistance, inductance, and capacitance) of elements (a resistor (R_{S}), an inductor (L_{S}), and capacitors (C_{S1} and C_{S2})) included in the variable control module by applying a variable control signal to the variable control module so that the simulated circuit (S) may be realized as the equivalent circuit diagram of the transducer circuit (T).

By controlling the element values of the variable control module, the simulated circuit (S) having circuit characteristic the same as the transducer circuit (T) may be realized.

That is, the element values (e.g., resistance, inductance, and capacitance) of the elements (e.g., R_{S}, L_{S}, C_{S1}, C_{S2}) of the variable control module may be controlled to be identical to element values of elements (R_{T}, L_{T}, C_{T1}, C_{T2}) of the transducer module in FIG. 7 so that the simulated circuit (S) having circuit characteristic the same as the transducer circuit (T) may be realized.

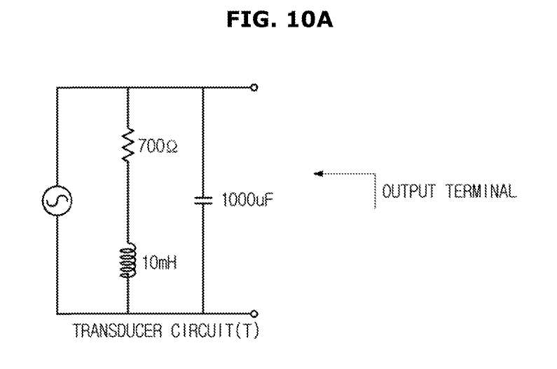

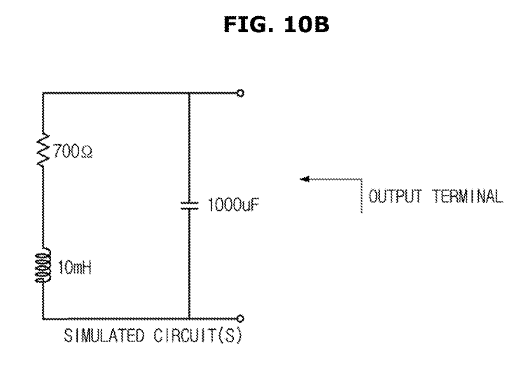

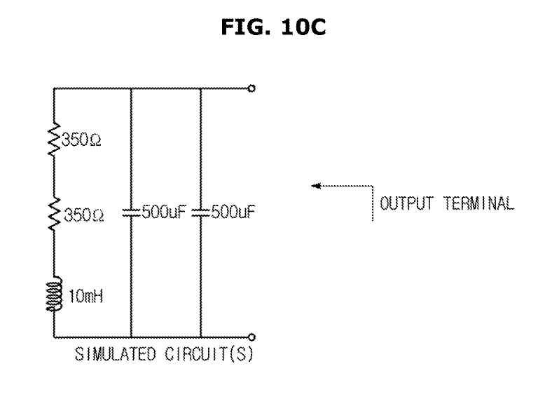

The variable control module may be realized in a configuration of FIG. 9, but is not limited thereto. The variable control module may be realized in various configurations having circuit characteristics the same as the transducer module. Likewise, the simulated circuit (S) containing the variable control module may be provided with elements arranged to be in an identical configuration to that of the transducer circuit (T) of FIG. 8, as well as provided with a circuit having circuit characteristics the same as the transducer circuit (T). FIG. 10A is an example of an equivalent circuit diagram of a transducer circuit (T), and FIGS. 10B and 10C are examples of an equivalent circuit diagram of a simulated circuit (S).

Referring to FIG. 10A, the transducer circuit (T) may have circuit characteristics of resistance of 700Ω, inductance of 10 mH, and capacitance of 1000 uF measured from an output terminal, the simulated circuit (S) may be realized by of a resistor of 700Ω, an inductor of 10 mH, and a capacitor of 1000 uF, as illustrated in FIG. 10B, or by two resistors of 350Ω connected in series, an inductor of 10 mH, and two capacitors of 500 uF connected in parallel, as illustrated in FIG. 10C. In addition, a simulated circuit (S) may be realized in various configurations having circuit characteristics the same as a transducer circuit (T). A circuit in FIG. 10B and a circuit in FIG. 10C may be equivalent with each other.

Referring to FIG. 8 again, the simulated circuit (S) may include the variable control module supplied with a voltage (V_{Bias} and C_{B} in FIG. 8) so that the simulated circuit (S) may output noise signals generated by the variable control module including the resistor (R_{S}), the inductor (L_{S}), and the capacitors (C_{S1} and C_{S2}) and other elements (R_{B} and C_{B}), hereinafter referred to as “power terminal.” The noise signals may be the same as noise signals output from the transducer circuit (T), and may be applied to the low-noise amplifier circuit (AMP_{S}).

The simulated circuit (S) may not output ultrasonic signals because the simulated circuit (S) may not include a transducer module receiving ultrasonic waves, unlike the transducer circuit (T).

Referring to FIG. 3 again, the probe module (PM) may include the low-noise amplifier circuits (AMP_{T} and AMP_{S}), and the low-noise amplifier circuits (AMP_{T} and AMP_{S}) may reduce a noise included in output signals of the transducer circuit (T) and the simulated circuit (S) and may include an esaki diode amplifier, a parametric amplifier, a maser amplifier, etc.

The low-noise amplifier circuit (AMP_{T}) connected to the transducer circuit (T) may output an ultrasonic signal (U′) and a noise signal (N′), obtained by performing a predetermined noise signal reduction process on an ultrasonic signal (U) and a noise signal (N) output from the transducer circuit (T).

The low-noise amplifier circuit (AMP_{S}) connected to the simulated circuit (S) may output a noise signal (N′), obtained by performing a predetermined noise signal reduction process on the noise signal (N) output from the simulated circuit (S).

However, the low-noise amplifier circuits (AMP_{T} and AMP_{S}) may be omitted depending on an embodiment.

The differential amplifier circuit (D) may amplify a voltage difference between two input signals provided thereto. Output signals of the differential amplifier circuit (D) may be proportional to a difference between two input signals. When the low-noise amplifier circuits (AMP_{T} and AMP_{S}) are connected to output terminals of the transducer circuit (T) and the simulated circuit (S), respectively, the differential amplifier circuit (D) may amplify a voltage difference (U′) between output signals (U′ and N′) output from the low-noise amplifier circuit (AMP_{T}) and output signals (N′) output from the low-noise amplifier circuit (AMP_{S}), and may output the voltage difference (U′).

When the low-noise amplifier circuits (AMP_{T} and AMP_{S}) are omitted, the differential amplifier circuit (D) may amplify a voltage difference (U) between the ultrasonic signal (U) and the noise signal (N) output from the transducer circuit (T), and the noise signal (N) output from the simulated circuit (S), may output the voltage difference (U), and may apply the voltage difference (U) to the ultrasonic receiving controller 410.

Output of the differential amplifier circuit (D) may be expressed as Equation 1.

Vₒᵤₜ=A_{d}(Vᵢₙ⁺−Vᵢₙ⁻) [Equation 1]

Herein, Vₒᵤₜ may represent an output signal of the differential amplifier circuit (D), Ad may represent a differential gain, Vᵢₙ⁺ may represent an input signal provided output from the low-noise amplifier circuit (AMP_{T}) connected to an output terminal of the transducer circuit (T), and Vᵢₙ⁻ may represent an input signal provided output from the low-noise amplifier circuit (AMP_{S}) connected to an output terminal of the simulated circuit (S).

A signal output from the differential amplifier circuit (D) may be applied to the ultrasonic receiving controller 410. The ultrasonic receiving controller 410 may produce an ultrasonic signal reconstructed based on a signal output from the probe module (PM) according to a command of the system controller 540. The reconstructed ultrasonic signal may be supplied to the image processor 510 and may be used to produce an ultrasonic image by the image processor 510. In this case, the ultrasonic receiving controller 410 may determine whether to receive an ultrasonic signal output from the transducer circuit (T) in the probe module (PM) according to the control signal (T_EN).

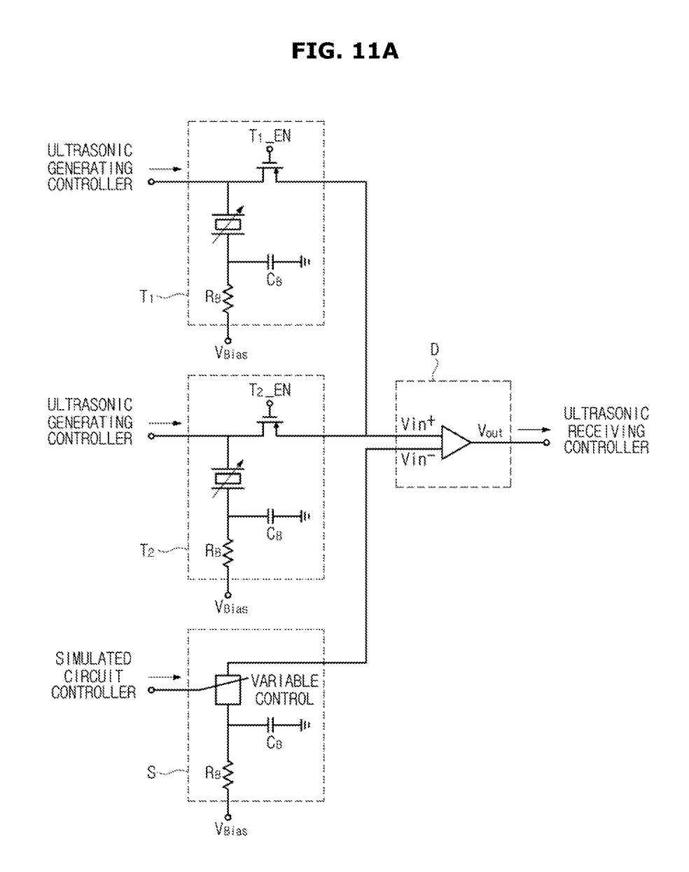

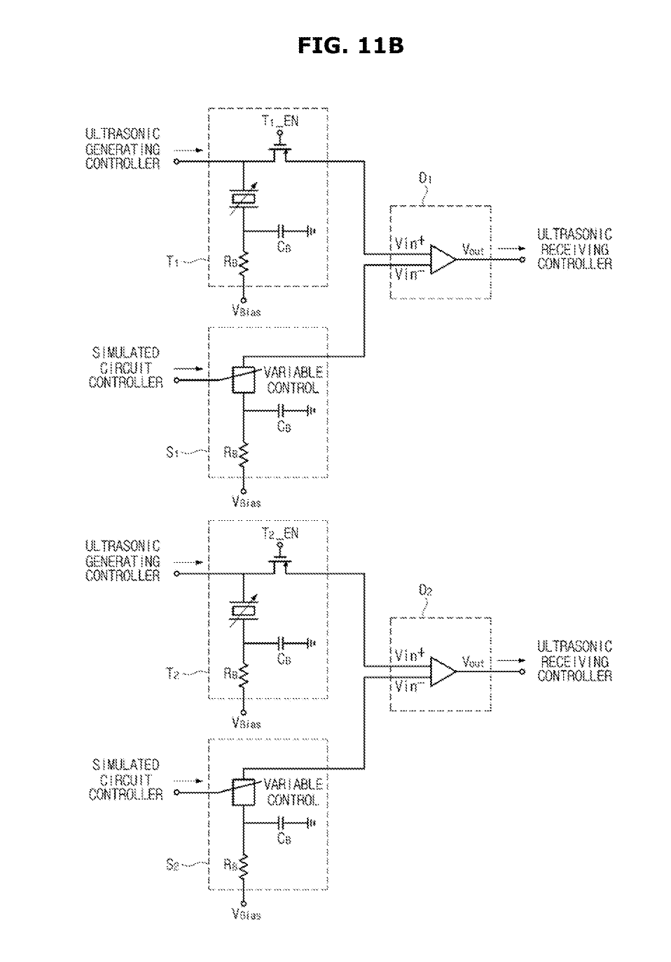

A plurality of transducer circuits (T) may be connected to each other. Each of transducer circuits (T₁ and T₂) may emit ultrasonic waves according to a control signal of the ultrasonic generating controller 420, and may output a plurality of ultrasonic signals and noise signals by receiving the ultrasonic waves according to a control signal of the ultrasonic receiving controller 410. FIGS. 11A and 11B are circuit diagrams of a probe module including a plurality of transducer circuits (T₁ and T₂).

In accordance with an exemplary embodiment, as illustrated in FIG. 11A, a single simulated circuit controller 430 may allow the simulated circuit (S) to have circuit characteristics the same as the plurality of transducer circuits (T₁ and T₂) by controlling element values of a variable control module in the simulated circuit (S).

In accordance with another exemplary embodiment, as illustrated in FIG. 11B, a plurality of simulated circuits (S₁ and S₂) may be connected to a plurality of transducer circuits (T₁ and T₂), respectively. In this case, the simulated circuit controller 430 may allow the simulated circuit (S) to have circuit characteristic the same as the plurality of transducer circuits (T₁ and T₂) by controlling element values of a variable control module in the simulated circuits (S₁ and S₂).

In the above-described exemplary embodiments, several components among components constituting the transducer circuit (T), the simulated circuit (S), the low-noise amplifier circuits (AMP_{T} and AMP_{S}), and the differential amplifier circuit (D) may be implemented by a type of module. The term “module” may represent software, or hardware, such as a field programmable gate array (FPGA), or an application specific integrated circuit (ASIC). The module may perform predetermined functions. However, the module is not limited to software or hardware. Further, the module may be constructed to exist in an addressable storage module, or to operate one or more processors.

For instance, the module may include elements (e.g., software elements, object-oriented software elements, class elements, and task elements), processors, functions, properties, procedures, subroutines, segments of a program code, drivers, firmware, a microcode, a circuit, data, a database, data structures, tables, arrays, and variables. Herein, functions provided by components and modules may be provided by a smaller number of combined larger components and modules, or by a larger number of divided smaller components and modules. In addition, the components and modules may be realized to operate one or more central processing units (CPUs) in a device.

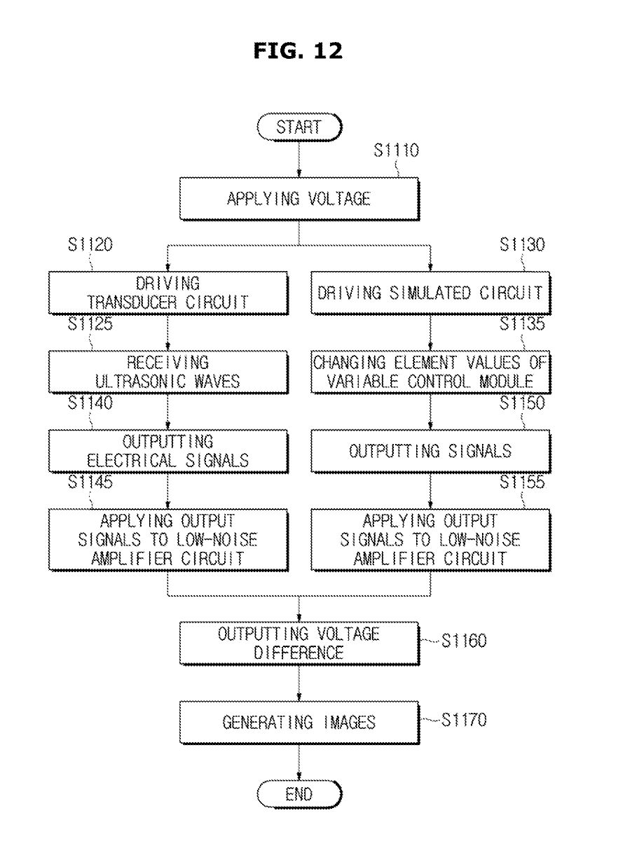

Hereinafter, a control method of an ultrasonic imaging apparatus will be described with reference to FIG. 12. FIG. 12 is a control flowchart of an ultrasonic imaging apparatus according to an exemplary embodiment. The control method of the ultrasonic imaging apparatus may be performed independently by the ultrasonic probe (P) or may be performed by the system controller 540 of the main body (M).

The ultrasonic imaging apparatus according to an exemplary embodiment may apply a voltage (V_{Bias}) to the probe module (PM) of the ultrasonic probe (P) in operation S1110. The probe module (PM) may include the transducer circuit (T), the simulated circuit (S), the low-noise amplifier circuits (AMP_{T} and AMP_{S}), and the differential amplifier circuit (D) so that the voltage (V_{Bias}) may applied to the transducer circuit (T), the simulated circuit (S), the low-noise amplifier circuits (AMP_{T} and AMP_{S}), and the differential amplifier circuit (D), respectively.

When applying the voltage, the transducer circuit (T) and the simulated circuit (S) may be driven in operations S1120 and S1130. When the transducer circuit (T) is driven in operation S1120, the ultrasonic imaging apparatus may control the ultrasonic probe (P) so that the transducer circuit (T) may receive ultrasonic waves reflected from an object (ob) in operation S1125. The ultrasonic waves reflected from the object (ob) may be a signal reflected by ultrasonic waves emitted from the ultrasonic probe (P) of the ultrasonic imaging apparatus.

When the ultrasonic probe (P) receives the ultrasonic waves in operation S1125, the ultrasonic waves received in the transducer circuit (T) may be converted into electrical signals and output in operation S1140.

When the simulated circuit (S) is driven in operation S1130, the ultrasonic imaging apparatus may control element values of the variable control module by applying a variable control signal to the simulated circuit (S) in operation S1135. In this case, the simulated circuit (S) may include elements (e.g., resistor, inductor, capacitor) having circuit characteristics the same as the transducer circuit (T). For example, when the transducer circuit (T) may include resistor (R_{B}) and capacitor (C_{B}), in which the resistor (R_{B}) may be connected between the transducer module and the power (V_{Bias}), and the capacitor (C_{B}) may be connected to a node between the transducer module and the resistor (R_{B}), and ground, the simulated circuit (S) may include resistor (R_{B}) and capacitor (C_{B}), in which the resistor (R_{B}) may be connected between the variable control module and a power (V_{Bias}), and the capacitor (C_{B}) may be connected to a node between the variable control module, and ground.

The simulated circuit (S) may be provided with elements arranged in an identical configuration to that of the transducer circuit (T), as well as provided with a circuit having circuit characteristic the same as the transducer circuit (T).

Output signals (i.e., an ultrasonic signal and a noise signal) of the transducer circuit (T) and an output signal (i.e., a noise signal) of the simulated circuit (S) may be applied to the low-noise amplifier circuits (AMP_{T} and AMP_{S}), respectively, and may pass through the low-noise amplifier circuits (AMP_{T} and AMP_{S}) in operations S1145 and S1155. The ultrasonic imaging apparatus may calculate a voltage difference between a signal output from the low-noise amplifier circuit (AMP_{T}) connected to the transducer circuit (T) and a signal output from the low-noise amplifier circuit (AMP_{S}) connected to the simulated circuit (S), and may output the voltage difference in operation S1160. In this case, the ultrasonic imaging apparatus may generate a reconstructed ultrasonic signal based on the voltage difference, and may produce an ultrasonic image based on the reconstructed ultrasonic signal in operation S1170. As mentioned above, by implementing the simulated circuit (S) having circuit characteristic the same as the transducer circuit (T) and by controlling the transducer circuit (T) and the simulated circuit (S), a noise signal, which may be generated by a power terminal of the transducer circuit (T) and included in a signal output from the transducer circuit (T), may be removed.

The simulated circuit (S) may be electrically equivalent to the transducer circuit (T) in a steady state of not receiving ultrasonic signals, and may be applied with a voltage (V_{Bias}) identical to that of the transducer circuit (T). Therefore, the simulated circuit (S) may control so that the differential amplifier circuit (D) may calculate a difference between an output signal of the transducer circuit (T) and an output signal of the simulated circuit (S), and thus may output an ultrasonic signal from which a noise signal is removed.

The control method of the ultrasonic imaging apparatus may be implemented in a computer-readable medium storing computer-readable codes. The computer-readable medium may be any medium that can store data readable by a computer system. For example, the computer-readable medium may be a read only memory (ROM), a random access memory (RAM), a magnetic tape, a magnetic disk, a flash memory, and an optical data storage medium. In addition, the medium may be distributed to computer systems over a network, in which computer-readable codes may be stored and executed in a distributed manner.

As described above, according to the ultrasonic imaging apparatus, the ultrasonic probe (P), and the control method of the ultrasonic imaging apparatus according to the exemplary embodiments, an ultrasonic signal having improved quality may be output without performing an additional signal processing on a noise signal, and a clearer ultrasonic image may be obtained based on the output ultrasonic signal.

The foregoing exemplary embodiments and advantages are merely exemplary and are not to be construed as limiting. The present teaching can be readily applied to other types of apparatuses. The description of the exemplary embodiments is intended to be illustrative, and not to limit the scope of the claims, and many alternatives, modifications, and variations will be apparent to those skilled in the art.

What is claimed is:

1. An ultrasonic probe comprising:
a transducer circuit configured to receive ultrasonic waves reflected from an object and output an ultrasonic signal based on the ultrasonic waves;
a simulated circuit having circuit characteristics substantially the same as the transducer circuit and configured to output a noise signal; and
a differential amplifier circuit configured to output a voltage difference between the ultrasonic signal and the noise signal.

2. The ultrasonic probe of claim 1, wherein
the transducer circuit and the simulated circuit are supplied with a voltage from a same power source.

3. The ultrasonic probe of claim 1, wherein
the simulated circuit comprises one or more electrical elements, and is configured to output the noise signal based on a variable control configured to control at least one value of the one or more electrical elements.

4. The ultrasonic probe of claim 1, wherein
the differential amplifier circuit is configured to amplify the voltage difference between the ultrasonic signal of the transducer circuit and the noise signal of the simulated circuit.

5. The ultrasonic probe of claim 1, further comprising:
a low-noise amplifier circuit configured to reduce at least one of noise included in the ultrasonic signal of the transducer circuit and the noise signal of the simulated circuit.

6. The ultrasonic probe of claim 1, wherein
the transducer circuit comprises a transducer module configured to emit the ultrasonic waves to the object and receive the ultrasonic waves reflected from the object.

7. An ultrasonic imaging apparatus comprising:
a display;
an ultrasonic probe configured to output a voltage difference between an ultrasonic signal of a transducer circuit and a noise signal of a simulated circuit, the transducer circuit receiving ultrasonic waves reflected from an object and outputting the ultrasonic signal based on the ultrasonic waves, and the simulated circuit having circuit characteristics substantially the same as the transducer circuit and outputting the noise signal; and
a controller configured to control the ultrasonic probe, filter noise from the ultrasonic signal based on the noise signal, generate an ultrasonic image from the filtered ultrasonic signal, and control the display to display the ultrasonic image.

8. The ultrasonic imaging apparatus of claim 7, wherein
the controller comprises a simulated circuit controller configured to control the simulated circuit.

9. The ultrasonic imaging apparatus of claim 7, further comprising:
a power source configured to supply a voltage to the transducer circuit and the simulated circuit.

10. The ultrasonic imaging apparatus of claim 7, wherein
the simulated circuit comprises one or more electrical elements, and is configured to output the noise signal based on a variable control configured to control at least one value of the one or more electrical elements.

11. The ultrasonic imaging apparatus of claim 7, further comprising:
a differential amplifier circuit configured to amplify the voltage difference.

12. The ultrasonic imaging apparatus of claim 7, further comprising:
a low-noise amplifier circuit configured to reduce at least one of noise included in the first ultrasonic signal of the transducer circuit and the noise signal of the simulated circuit.

13. The ultrasonic imaging apparatus of claim 7, wherein the controller comprises:
an ultrasonic generating controller configured to control the transducer circuit to emit the ultrasonic waves; and
an ultrasonic receiving controller configured to generate a reconstructed ultrasonic signal based on the voltage difference.

14. The ultrasonic imaging apparatus of claim 7, wherein
the transducer circuit comprises a transducer module configured to emit the ultrasonic waves to the object and receive the ultrasonic waves from the object.

15. The ultrasonic imaging apparatus of claim 13, wherein the controller further comprises:
an imaging processor configured to generate the ultrasonic image based on the reconstructed ultrasonic signal.

16. A method of controlling an ultrasonic imaging apparatus, the method comprising:
supplying a voltage to a transducer circuit and a simulated circuit, the simulated circuit having circuit characteristics substantially the same as the transducer circuit;
receiving ultrasonic waves reflected from an object;
outputting, by the transducer circuit, an ultrasonic signal based on the ultrasonic waves, and outputting, by the simulated circuit, a noise signal;
outputting a voltage difference between the ultrasonic signal of the transducer circuit and the noise signal of the simulated circuit; and
filtering noise from the ultrasonic signal based on the noise signal and generating an ultrasonic image from the filtered ultrasonic signal.

17. The method of claim 16, further comprising:
controlling at least one value of one or more electrical elements of the simulated circuit.

18. The method of claim 16, further comprising:
emitting the ultrasonic waves to the object through the transducer; and
receiving the ultrasonic waves reflected from the object.

19. The method of claim 16, wherein
the outputting the voltage difference comprises amplifying the voltage difference.
